(19) United States
(12) Patent Application Publication
(10) Pub. No.: US 2010/0261239 A1
(43) Pub. Date: Oct. 14, 2010

(54) METABOLICALLY ENGINEERED MICROORGANISM USEFUL FOR THE PRODUCTION OF 1,2-PROPANEDIOL

(75) Inventors: Philippe Soucaille, Deyme (FR); Francois Voelker, Montrond Les Bains (FR); Rainer Figge, Riom (FR)

Correspondence Address: Baker Donelson Bearman, Caldwell & Berkowitz, PC, 920 Massachusetts Ave, NW, Suite 900, Washington, DC, 20001, US

(73) Assignee: Metalbolic Explorer, Saint Beauzire (FR)

(21) Appl. No.: 12/532,423

(22) PCT Filed: Mar. 21, 2008
(86) PCT No.: PCT/EP08/53438

§ 371 Date: Sep. 22, 2009

(30) Foreign Application Priority Data
Mar. 23, 2007 (IB) PCT/IB2007/001675

(51) Int. Cl. C12P 7/18; C12N 1/21

(52) U.S. Cl. 435/158; 435/252.3; 435/252.33

(57) ABSTRACT

Microorganism useful for the production of 1,2-propanediol from a carbon source, wherein said microorganism is characterized by:
an improved activity of the biosynthesis pathway from dihydroxyacetone phosphate to 1,2-propanediol, and
an attenuated activity of the glyceraldehyde 3-phosphate dehydrogenase
The invention is also related to a method for producing 1,2-propanediol by fermentation with a microorganism according to the invention.

The present invention concerns a metabolically engineered micro-organism and its use for the preparation of 1,2-propanediol.

1,2-propanediol or propylene glycol, a C3 dialcohol, is a widely-used chemical. It is a component of unsaturated polyester resins, liquid detergents, coolants, anti-freeze and de-icing fluids for aircraft. Propylene glycol has been increasingly used since 1993-1994 as a replacement for ethylene derivatives, which are recognised as being more toxic than propylene derivatives.

1,2-propanediol is currently produced by chemical means using a propylene oxide hydration process that consumes large amounts of water. Propylene oxide can be produced by either of two processes, one using epichlorhydrin, and the other hydroperoxide. Both routes use highly toxic substances. In addition, the hydroperoxide route generates by-products such as tert-butanol and 1-phenyl ethanol. For the production of propylene to be profitable, a use must be found for these by-products. The chemical route generally produces racemic 1,2-propanediol, whereas each of the two stereoisomers (R)1,2-propanediol and (S)1,2-propanediol are of interest for certain applications.

The disadvantages of the chemical processes for the production of 1,2-propanediol make biological synthesis an attractive alternative. Two routes have been characterized for the natural production of 1,2-propanediol from sugars by microorganisms.

In the first route 6-deoxy sugars (e.g. L-rhamnose or L-fucose) are cleaved into dihydroxyacetone phosphate and (S)-lactaldehyde, which can be further reduced to (S)-1,2-propanediol (Badia et al, 1985). This route is functional in E. coli, but can not yield an economically feasible process due to the elevated cost of the deoxyhexoses.

The second route is the metabolism of common sugars (e.g. glucose or xylose) through the glycolysis pathway followed by the methylglyoxal pathway. Dihydroxyacetone phosphate is converted to methylglyoxal that can be reduced either to lactaldehyde or to acetol. These two compounds can then undergo a second reduction reaction yielding 1,2-propanediol. This route is used by natural producers of (R)-1,2-propanediol, such as Clostridium sphenoides and Thermoanaerobacter thermosaccharolyticum. Clostridium sphenoides has been used to produce 1,2-propanediol at a titer of 1.58 g/l under phosphate limited conditions (Tran Din and Gottschalk, 1985). Thermoanaerobacter thermosaccharolyticum has also been investigated for the production of 1,2-propanediol (Cameron and Cooney, 1986, Sanchez-Rivera et al, 1987). The best performances obtained were a titer of 9 g/l and a yield from glucose of 0.2 g/g. However, the improvement of the performances obtained with these organisms is likely to be limited due to the shortage of available genetic tools.

### PRIOR ART

Cameron et al (1998) have investigated the use of E. coli as a platform for metabolic engineering for the conversion of sugars to 1,2-propanediol. Their theoretical analysis showed that the upper limit of the realistic product yield (considering mass balances and production of energy for growth) is significantly different depending on the culture conditions. Under anaerobic conditions, acetate will be produced as a by-product in order to recycle the reduced co-factors and the best yield shall be limited to 1 mole of 1,2-propanediol per mole of glucose (0.42 g/g). Under aerobic conditions, recycling of co-factors shall be ensured by the respiratory chain using oxygen as terminal electron acceptor and it could become possible to produce 1,2-propanediol without the production of by-products. Under these conditions, yield could reach at best 1.42 mol/mol (0.6 g/g). Considering the maximum titer of 1,2-propanediol, Cameron et al discussed its dependence on product and by-product toxicity. 1,2-propanediol is significantly less toxic than 1,3-propanediol and E. coli exhibits a residual growth rate of 0.5 h⁻¹ with 100 g/l 1,2-propanediol. The inhibition of growth is more likely to be due to the by-product acetate that is known to be highly growth inhibiting. Development of an anaerobic process for the production of 1,2-propanediol with high titers and yields will have to address the acetate issue. Conversion of acetate into acetone, which is less inhibitory and easily removed in situ has been proposed (WO 2005/073364).

Several investigations for genetic modifications of E. coli in order to obtain a 1,2-propanediol producer using simple carbon sources have been done by the group of Cameron (Cameron et al, 1998, Altaras and Cameron, 1999, Altaras and Cameron, 2000) and the group of Bennett (Huang et al, 1999, Berrios-Rivera et al, 2003). These studies rely on the one hand on the expression of one or several enzymatic activities in the pathway from dihydroxyacetone phosphate to 1,2-propanediol and on the other hand on the removal of NADH and carbon consuming pathways in the host strain. The best results obtained by the group of Cameron are production of 1.4 g/l 1,2-propanediol in anaerobic flask culture with a yield of 0.2 g/g of glucose consumed. When extrapolated in anaerobic fed-batch fermenter, the production was 4.5 g/l 1,2-propanediol with a yield of 0.19 g/g from glucose, far from their theoretical expectations. These performances have been obtained with the overexpression of the methylglyoxal synthase gene of E. coli (mgs), the glycerol dehydrogenase gene of E. coli (gldA) and the 1,2-propanediol oxidoreductase gene of E. coli (fucO) in a strain lacking the gene coding for lactate dehydrogenase (ldhA). Results obtained with the same approach but with lower titers and yields are also described in the patents U.S. Pat. No. 6,087,140, U.S. Pat. No. 6,303,352 and WO 98/37204.

The group of Bennett also used an E. coli host strain lacking ldhA for the overexpression of the mgs gene from Clostridium acetobutylicum and the gldA gene from E. coli. Flask cultures under anaerobic conditions gave a titer of 1.3 g/l and a yield of 0.12 g/g whereas microaerobic cultures gave a titer of 1.4 g/l with a yield of 0.13 g/g.

At this stage, all these results are not better than those obtained with the species T. thermosaccharolyticum.

The catabolism of glucose trough the glycolysis pathway in E. coli results in two triose phosphate molecules, dihydroxyacetone phosphate (DHAP) and glyceraldehyde 3 phosphate, after the cleavage of fructose 1,6 bisphosphate. These two triose phosphate molecules can be interconverted by the triose phosphate isomerase activity. It is generally recognized that DHAP is converted to GA3P and the two GA3P originating from glucose are further catabolized.

The glyceraldehyde 3-phosphate dehydrogenase, also called GAPDH, is one of the key enzymes involved in the glycolytic conversion of glucose to pyruvic acid. GAPDH catalyzes the following reaction:

Glyceraldehyde 3-phosphate+phosphate+NAD⁺→1,3-biphosphoglycerate+NADH+H⁺

The gene encoding this enzyme was cloned in 1983 in E. coli (Branlant et al., Gene, 1983) and named “gap”. Later another gene encoding a product having the same enzymatic activity was identified and named gapB (Alefounder et al., Microbiol., 1987). Characterization of E. coli strains with deleted gapA and gapB genes have shown that gapA is essential for glycolysis although gapB is dispensable (Seta et al., J. Bacter., 1997). A microorganism with a down regulated gapA gene was reported in patent application WO 2004/033646 for the production of 1,3-propanediol from glucose by fermentation.

The inventors of the present application have shown that 2 factors in combination are required to obtain an increase of the 1,2-propanediol yield:
an improved activity of the biosynthesis pathway of 1,2-propanediol, and
an attenuation of the GAPDH activity.

The inventors demonstrate also that increasing intracellular phosphoenolpyruvate concentration or using an alternative sugar transport system can further boost the 1,2-propanediol production by fermentation of a micro-organism.

### DESCRIPTION OF THE INVENTION

The invention is related to a microorganism useful for the production of 1,2-propanediol from a carbon source, wherein said microorganism is characterized by:
a) an improved activity of the biosynthesis pathway from dihydroxyacetone phosphate to 1,2-propanediol, and
b) an attenuated activity of the glyceraldehyde 3-phosphate dehydrogenase

The improved activity of the biosynthesis pathway from DHAP to 1,2-propanediol is obtained by increasing the activity of at least one enzyme involved in said biosynthetic pathway. This can be obtained by increasing the expression of the gene coding for said enzyme and in particular the expression of at least one gene selected among mgsA, yqhD, yafB, ycdW, yqhE, yeaE, yghZ, yajO, tas, ydjG, ydbC, gldA and fucO. Preferentially, the expression of the three genes mgsA, yqhD and gldA is increased.

In a further aspect of the invention, the Entner-Doudoroff pathway is eliminated by deleting either the edd or eda gene or both. Furthermore, the synthesis of unwanted by-products is attenuated by deleting the genes coding for enzymes involved in synthesis of lactate from methylglyoxal (such as gloA, aldA, aldB), lactate from pyruvate (ldhA), formate (pflA, pflB), ethanol (adhE) and acetate (ackA, pta, poxB).

The glyceraldehyde 3 phosphate activity is attenuated in order to redirect a part of the available glyceraldehyde 3 phosphate toward the synthesis of 1,2-propanediol via the action of the enzyme triose phosphate isomerase. The yield of 1,2-propanediol over glucose can then be greater than 1 mole/mole. However, due to the reduced production of phosphoenolpyruvate (PEP), the PEP-dependent sugar import system will be negatively impacted. Therefore, in one aspect of the invention, the efficiency of the sugar import is increased, either by using a sugar import independent of PEP like the one encoded by galP, or by providing more PEP to the sugar-phosphotransferase system. This is obtained by eliminating the pathways consuming PEP like pyruvates kinases (encoded by the pykA and pykF genes) and/or by promoting the synthesis of PEP e.g. by overexpressing the ppsA gene coding for PEP synthase.

Additionally, it is valuable for the enzyme converting pyruvate into acetyl-coA to be resistant to high concentrations of NADH found under anaerobic conditions. This can be obtained by a specific mutation in the lpd gene. Finally, in order to spare NADH for the reduction of acetol into 1,2-propanediol, the arcA and the ndh genes can be deleted.

The microorganism used for the preparation of 1,2-propanediol is selected among bacteria, yeasts and fungi, but is preferentially from the species Escherichia coli or Clostridium acetobutylicum.

It is also an object of the present invention to provide a process for the production of 1,2-propanediol by cultivating the modified microorganism in an appropriate growth medium and by recovering and purifying the 1,2-propanediol produced.

### BRIEF DESCRIPTION OF THE DRAWINGS

The accompanying drawing that is incorporated in and constitutes a part of this specification exemplifies the invention and together with the description, serves to explain the principles of this invention.

FIG. 1 depicts the genetic engineering of central metabolism in the development of a 1,2-propanediol production system from carbohydrates.

### DETAILED DESCRIPTION OF THE INVENTION

As used herein the following terms may be used for interpretation of the claims and specification.

According to the invention the terms ‘culture’, ‘growth’ and ‘fermentation’ are used interchangeably to denote the growth of bacteria in an appropriate growth medium containing a simple carbon source.

The term ‘carbon source’ according to the present invention denotes any source of carbon that can be used by those skilled in the art to support the normal growth of a micro-organism, and which can be hexoses, pentoses, monosaccharides, disaccharaides, oligosaccharides, starch or its derivatives, hemicelluloses, glycerol and combinations thereof.

The term “useful for the production of 1,2-propanediol” denotes that the microorganism produces said product of interest, preferably by fermentation. Fermentation is a classical process that can be performed under aerobic, microaerobic or anaerobic conditions.

The phrase “attenuation of the activity of an enzyme” refers to a decrease of the activity of the enzyme of interest in the modified strain compared to the activity in the initial strain before any modification. The man skilled in the art knows numerous means to obtain this result. Possible examples include:
Introduction of a mutation into the gene, decreasing the expression level of this gene, or the level of activity of the encoded protein.
Replacement of the natural promoter of the gene by a low strength promoter, resulting in a lower expression.
Use of elements destabilizing the corresponding messenger RNA or the protein.
Deletion of the gene if no expression at all is needed.

The term “expression” refers to the transcription and translation of a gene sequence leading to the generation of the corresponding protein product of the gene.

Advantageously, the activity of the glyceraldehyde 3-phosphate dehydrogenase is less than 30% of the activity observed in an unmodified strain under the same conditions, more preferably less than 10%.

The term “improved activity of the biosynthesis pathway from dihydroxyacetone phosphate to 1,2-propanediol” means that at least one of the enzymatic activities involved in the pathway is improved (see below).

Advantageously, the microorganism of the invention is genetically modified to increase the activity of at least one enzyme involved in the biosynthetic pathway from dihydroxyacetone phosphate to 1,2-propanediol.

Preferentially, the increase of the activity of an enzyme is obtained by increasing the expression of the gene coding for said enzyme.

To obtain an overexpression of a gene of interest, the man skilled in the art knows different methods such as:
Replacement of the endogenous promoter with a stronger promoter
Introduction into the microorganism of an expression vector carrying said gene of interest.
Introducing additional copies of the gene of interest into the chromosome

Several techniques are currently used for introducing DNA into a bacterial strain. A preferred technique is electroporation, which is well known to those skilled in the art.

Advantageously, at least one gene of interest is overexpressed, selected among: mgsA, yafB, yeaE, yghZ, yqhE, yqhD, ydhF, ycdW, yajO, ydjG, ydbC, tas, gldA and fucO.

The mgsA gene codes for methylglyoxal synthase catalysing the conversion of DHAP into methylglyoxal. The genes yafB, yeaE, yghZ, yqhE, yqhD, ydhF, ycdW, yajO, ydjG, ydbC, tas encode enzymatic activities able to convert methylglyoxal into acetol. The gldA gene encodes glycerol dehydrogenase, which catalyses the conversion of acetol into 1,2-propanediol. The fucO gene encodes 1,2-propanediol oxidoreductase catalysing the conversion of lactaldehyde into 1,2-propanediol.

A preferred microorganism harbours modifications leading to the overexpression of three genes of particular interest: mgsA, yqhD and gldA.

Preferentially, in the microorganism according to the invention, at least one gene involved in the Entner-Doudoroff pathway is attenuated. The Entner-Doudoroff pathway provides an alternative way to degrade glucose to glyceraldehyde-3-phosphate and pyruvate besides glycolysis. The attenuation of the Entner-Doudoroff pathway assures that most or at best all glucose is degraded via glycolysis and be used for the production of 1,2-propanediol.

In particular at least one of the two genes of this pathway edd or eda is attenuated.

The term ‘attenuation of the expression of a gene’ according to the invention denotes the partial or complete suppression of the expression of a gene, which is then said to be ‘attenuated’. This suppression of expression can be either an inhibition of the expression of the gene, the suppression of an activating mechanism of the gene, a deletion of all or part of the promoter region necessary for the gene expression, or a deletion in the coding region of the gene. Preferentially, the attenuation of a gene is essentially the complete deletion of that gene, which gene can be replaced by a selection marker gene that facilitates the identification, isolation and purification of the strains according to the invention. A gene is preferentially inactivated by the technique of homologous recombination as described in Datsenko, K. A. & Wanner, B. L. (2000) “One-step inactivation of chromosomal genes in Escherichia coli K-12 using PCR products”. Proc. Natl. Acad. Sci. USA 97: 6640-6645.

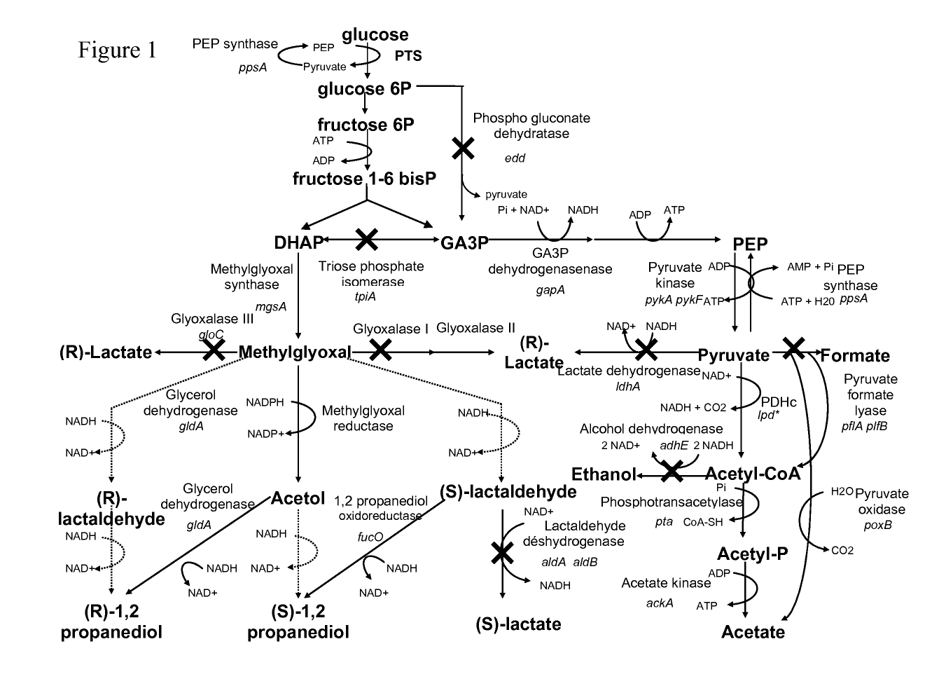

Preferentially, in the microorganism according to the invention, at least one enzyme involved in the conversion of methylglyoxal into lactate is attenuated. The purpose of this attenuation is that the available methylglyoxal is used by the cell machinery essentially for the synthesis of 1,2-propanediol (see FIG. 1).

Genes involved in the conversion of methylglyoxal into lactate are in particular:
Genes encoding for enzymes having glyoxalase activity, such as the gloA gene coding for glyoxalase I, catalysing the synthesis of lactoyl glutathione from methylglyoxal;
the aldA and aldB genes coding for a lactaldehyde dehydrogenase (catalysing the synthesis of (S) lactate from (S) lactaldehyde).
The expression of one or more of these genes is advantageously attenuated in the initial strain. Preferentially the gene gloA is completely deleted.

In the microorganism of the invention, it is preferable that at least one enzyme involved in the synthesis of by-products such as lactate, ethanol and formate is attenuated.

In particular, it is advantageous to attenuate the gene ldhA coding for lactate dehydrogenase catalysing the synthesis of lactate from pyruvate, and the gene adhE coding for alcohol-aldehyde dehydrogenase catalysing the synthesis of ethanol from acetyl-CoA.

Similarly, it is possible to force the micro-organism to use the pyruvate dehydrogenase complex to produce acetyl-CoA, CO2 and NADH from pyruvate, instead of acetyl-CoA and formate. This can be achieved by attenuating the genes pflA and pflB coding for pyruvate formate lyase.

In another specific embodiment of the invention, the synthesis of the by-product acetate is prevented by attenuating at least one enzyme involved in its synthesis. It is preferable to avoid such acetate synthesis to optimize the production of 1,2-propanediol.

To prevent the production of acetate, advantageously the expression of at least one gene selected among ackA, pta and poxB is attenuated. These genes all encode enzymes involved in the different acetate biosynthesis pathways (see FIG. 1).

Preferentially, in the microorganism according to the invention, the efficiency of sugar import is increased. A strong attenuation of the expression of the gapA gene resulting in a decrease of the carbon flux in the GAPDH reaction by more than 50%, this will result in the synthesis of less than 1 mole of phosphoenolpyruvate (PEP) per mole of glucose imported. PEP is required by the sugar-phosphotransferase system (PTS) normally used for the import of simple sugars into the cell, since import is coupled to a phospho-transfer from PEP to glucose yielding glucose-6-phosphate. Thus reducing the amount of PEP will negatively impact on sugar import.

In a specific embodiment of the invention, the sugar might be imported into the microorganism by a sugar import system independent of phosphoenolpyruvate. The galactose-proton symporter encoded by the gene galP that does not involve phosphorylation can be utilized. In this case the imported glucose has to be phosphorylated by glucose kinase encoded by the glk gene. To promote this pathway, the expression of at least one gene selected among galP and glk is increased. As a result the PTS becomes dispensable and may be eliminated by attenuating at least one gene selected among ptsH, ptsI or crr.

In another specific embodiment of the invention, the efficiency of the sugar-phosphotransferase system (PTS) is increased by increasing the availability of the metabolite phosphoenopyruvate. Due to the attenuation of the gapA activity and of the lower carbon flux toward pyruvate, the amount of PEP in the modified strain of the invention could be limited, leading to a lower amount of glucose transported into the cell.

Various means exist that may be used to increase the availability of PEP in a strain of microorganism. In particular, a mean is to attenuate the reaction PEP→pyruvate. Preferentially, at least one gene selected among pykA and pykF, coding for the pyruvate kinase enzyme, is attenuated in said strain to obtain this result. Another way to increase the availability of PEP is to favour the reaction pyruvate→PEP, catalyzed by the phosphoenolpyruvate synthase by increasing the activity of the enzyme. This enzyme is encoded by the ppsA gene. Therefore, preferentially in the microorganism, the expression of the ppsA gene is preferentially increased. Both modifications can be present in the microorganism simultaneously.

Especially under anaerobic or microaerobic conditions, it is advantageous that the pyruvate dehydrogenase complex (PDC), converting pyruvate into acetyl-coA has low sensitivity to inhibition by NADH. Lower sensitivity is defined with reference to the sensitivity of the unmodified enzyme. Such characteristic can be obtained by introducing a specific mutation in the lpd gene (coding for the sub-unit lipoamide dehydrogenase of the PDC) resulting in the replacement of alanine 55 in the protein sequence of the enzyme with the residue valine.

Under anaerobic or microaerobic conditions, availability of NADH for the reduction of the precursors into 1,2-propanediol is advantageously increased. This is obtained by alleviating the repression on the tricarboxylic acid cycle mediated by the global regulator ArcA (encoded by the arcA gene). NADH concentration in the cell can also be increased by inactivating the NADH dehydrogenase II encoded by the gene ndh. Therefore, preferably, at least one gene selected among arcA and ndh is attenuated.

Preferentially the microorganism according to the invention is selected among bacteria, yeasts or fungi. More preferentially, the microorganism is selected from the group consisting of Enterobacteriaceae, Bacillaceae, Clostridiaceae, Streptomycetaceae and Corynebacteriaceae. Even more preferentially, the microorganism is either Escherichia coli or Clostridium acetobutylicum.

Another object of the invention is a method for preparing 1,2-propanediol, wherein a microorganism such as described previously is grown in an appropriate growth medium containing a simple carbon source, and the produced 1,2-propanediol is recovered. The production of 1,2-propanediol is performed under aerobic, microaerobic or anaerobic conditions.

The culture conditions for the fermentation process can be readily defined by those skilled in the art. In particular, bacteria are fermented at temperatures between 20° C. and 55° C., preferably between 25° C. and 40° C., and preferably at about 35° C. for C. acetobutylicum and at about 37° C. for E. coli.

This process can be carried out either in a batch process, in a fed-batch process or in a continuous process.

‘Under aerobic conditions’ means that oxygen is provided to the culture by dissolving the gas into the liquid phase. This could be obtained by (1) sparging oxygen containing gas (e.g. air) into the liquid phase or (2) shaking the vessel containing the culture medium in order to transfer the oxygen contained in the head space into the liquid phase. Advantages of the fermentation under aerobic conditions instead of anaerobic conditions is that the presence of oxygen as an electron acceptor improves the capacity of the strain to produce more energy in form of ATP for cellular processes. Therefore the strain has its general metabolism improved.

Micro-aerobic conditions are defined as culture conditions wherein low percentages of oxygen (e.g. using a mixture of gas containing between 0.1 and 10% of oxygen, completed to 100% with nitrogen), is dissolved into the liquid phase.

Anaerobic conditions are defined as culture conditions wherein no oxygen is provided to the culture medium. Strictly anaerobic conditions are obtained by sparging an inert gas like nitrogen into the culture medium to remove traces of other gas. Nitrate can be used as an electron acceptor to improve ATP production by the strain and improve its metabolism.

The term ‘appropriate growth medium’ according to the invention denotes a medium of known molecular composition adapted to the growth of the micro-organism. For example a mineral culture medium of known set composition adapted to the bacteria used, containing at least one carbon source. In particular, the mineral growth medium for E. coli can thus be of identical or similar composition to M9 medium (Anderson, 1946, Proc. Natl. Acad. Sci. USA 32:120-128), M63 medium (Miller, 1992; A Short Course in Bacterial Genetics: A Laboratory Manual and Handbook for Escherichia coli and Related Bacteria, Cold Spring Harbor Laboratory Press, Cold Spring Harbor, N.Y.) or a medium such as that defined by Schaefer et al. (1999, Anal. Biochem. 270: 88-96), and in particular the minimum culture medium named MPG described below:

| | | |
|---|---|---|
| K₂HPO₄ | 1.4 | g/l |
| Nitrilo Triacetic Acid | 0.2 | g/l |
| trace element solution* | 10 | ml/l |
| (NH₄)₂SO₄ | 1 | g/l |
| NaCl | 0.2 | g/l |
| NaHCO₃ | 0.2 | g/l |
| MgSO₄ | 0.2 | g/l |
| glucose | 20 to 100 | g/l |
| NaNO₃ | 0.424 | g/l |
| thiamine | 10 | mg/l |
| FeSO₄, 7H₂O | 50 | mg/l |
| yeast extract | 4 | g/l |

*trace element solution: Citric acid 4.37 g/L, MnSO₄ 3 g/L, CaCl₂ 1 g/L, CoCl₂, 2H₂O 0.1 g/L, ZnSO₄, 7H₂O 0.10 g/L, CuSO₄, 5H₂O 10 mg/L, H₃BO₃ 10 mg/L, Na₂MoO₄ 8.31 mg/L.

The pH of the medium is adjusted to 7.4 with sodium hydroxide.

In a specific embodiment of the invention, the method is performed with a strain of E. coli grown in a medium containing a simple carbon source that can be arabinose, fructose, galactose, glucose, lactose, maltose sucrose or xylose. An especially preferred simple carbon source is glucose.

In another specific embodiment of the invention, the method is performed with a strain of C. acetobutylicum grown in a medium containing a simple or a complex carbon source.

The growth medium for can thus be of identical or similar composition to Clostridial Growth Medium (CGM, Wiesenborn et al., Appl. Environm. Microbiol., 54: 2717-2722) or a mineral growth medium as given by Monot et al. (Appl. Environm. Microbiol., 44: 1318-1324) or Vasconcelos et al. (J. Bacteriol., 176: 1443-1450).

The carbon source used for the culture of C. acetobutylicum is either a simple or a complex carbon. The simple carbon source can be arabinose, fructose, galactose, glucose, lactose, maltose sucrose or xylose. An especially preferred simple carbon source is glucose. The complex carbon source can be starch or hemicellulose. An especially preferred complex carbon source is starch.

Advantageously the recovered 1,2-propanediol is furthermore purified. The man skilled in the art knows various means for recovering and purifying the 1,2-propanediol.

The invention is described above, below and in the Examples with respect to E. coli. Thus the genes that can be attenuated, deleted or over-expressed for the initial and evolved strains according to the invention are defined mainly using the denomination of the genes from E. coli. However, this designation has a more general meaning according to the invention, and covers the corresponding genes in other micro-organisms. Using the GenBank references of the genes from E. coli, those skilled in the art can determine equivalent genes in other organisms than E. coli.

The means of identification of the homologous sequences and their percentage homologies are well-known to those skilled in the art, and include in particular the BLAST programmes that can be used on the website http://www.ncbi.nlm.nih.gov/BLAST/ with the default parameters indicated on that website. The sequences obtained can be exploited (aligned) using for example the programmes CLUSTALW (http://www.ebi.ac.uk/clustalw/), with the default parameters indicated on these websites.

The PFAM database (protein families database of alignments and hidden Markov models http://www.sanger.ac.uk/Software/Pfam/) is a large collection of alignments of protein sequences. Each PFAM makes it possible to visualise multiple alignments, view protein domains, evaluate distributions among organisms, gain access to other databases and visualise known protein structures.

COGs (clusters of orthologous groups of proteins http://www.ncbi.nlm.nih.gov/COG/) are obtained by comparing protein sequences derived from 66 fully sequenced unicellular genomes representing 44 major phylogenetic lines. Each COG is defined from at least three lines, making it possible to identify ancient conserved domains.

### REFERENCES IN THE ORDER OF CITATION IN THE TEXT

1. Badia J, Ros J, Aguilar J (1985), J. Bacteriol. 161: 435-437.
2. Tran Din K and Gottschalk G (1985), Arch. Microbiol. 142: 87-92
3. Cameron D C and Cooney C L (1986), Bio/Technology, 4: 651-654
4. Sanchez-Rivera F, Cameron D C, Cooney C L (1987), Biotechnol. Lett. 9: 449-454
5. Altaras N E and Cameron D C (1999), Appl. Environ. Microbiol. 65: 1180-1185
6. Cameron D C, Altaras N E, Hoffman M L, Shaw A J (1998), Biotechnol. Prog. 14: 116-125
7. Altaras N E and Cameron D C (2000), Biotechnol. Prog. 16: 940-946
8. Huang K, Rudolph F B, Bennett G N (1999), Appl. Environ. Microbiol. 65: 3244-3247
9. Berrios-Rivera S J, San K Y, Bennett G N (2003), J. Ind. Microbiol. Biotechnol. 30: 34-40
10. Branlant G, Flesch G, Branlant C (1983), Gene, 25: 1-7
11. Alefounder P R and Perham R N (1989), Mol. Microbiol., 3: 723-732
12. Seta F D, Boschi-Muller F, Vignais M L, Branlant G (1997), J. Bacteriol. 179: 5218-5221
13. Datsenko K A and Wanner B L (2000), Proc. Natl. Acad. Sci. USA 97: 6640-6645
14. Anderson E H (1946), Proc. Natl. Acad. Sci. USA 32:120-128
15. Miller (1992), A Short Course in Bacterial Genetics: A Laboratory Manual and Handbook for Escherichia coli and Related Bacteria, Cold Spring Harbor Laboratory Press, Cold Spring Harbor, N.Y.
16. Schaefer U, Boos W, Takors R, Weuster-Botz D (1999), Anal. Biochem. 270: 88-96
17. Wiesenborn D P, Rudolph R B, Papoutsakis E T (1987), Appl. Environ. Microbiol., 54: 2717-2722
18. Monot F, Martin J R, Petitdemange H, Gay R (1982), Appl. Environ. Microbiol. 44: 1318-1324
19. Vasconcelos I, Girbal L, Soucaille P (1994), J. Bacteriol. 176: 1443-1450
20. Lerner C G and Inouye M (1990), Nucleic Acids Res. 18: 4631

### EXAMPLES

### Example 1

### Construction of Modified Strains of E. coli MG1655 Ptrc16-gapA::cm (pME101VB01-yqhD-mgsA-gldA), E. coli MG1655 Ptrc16-gapA::cm (pME101VB01-yafB-mgsA-gldA) and E. coli MG1655 Ptrc16-gapA::cm (pME101VB01-mhE-mgsA-g/dA)

To increase the production of 1,2-propanediol different combinations of genes were expressed from the plasmid pME101VB01 using the trc promoter.

a) Construction of Modified Strains of E. coli MG1655 (pME101VB01-yqhD-mgsA-g/dA), MG1655 (pME101VB01-yafB-mgsA-gldA) and MG1655 (pME101VB01-mhE-mgsA-g/dA) Construction of Plasmid pME101VB01

The plasmid pME101VB01 was derived from plasmid pME101 and harbored a multiple cloning site containing recognition site sequences specific for the rare restriction endonucleases NheI, SnaBI, Pad, BglII, AvrII, SacII and AgeI following by the adc transcription terminator of Clostridium acetobutylicum ATCC 824.

For the expression from a low copy vector the plasmid pME101 was constructed as follows. The plasmid pCL1920 (Lerner & Inouye, 1990, NAR 18, 15 p 4631-GenBank AX085428) was PCR amplified using the oligonucleotides PME101F and PME101R and the BstZ17′-XmnI fragment from the vector pTrc99A (Amersham Pharmacia Biotech, Piscataway, N.J.) harboring the lad gene and the trc promoter was inserted into the amplified vector.

| | |
|---|---|
| PME101F (SEQ ID NO 1): | ccgacagtaagacgggtaagcctg |
| PME101R (SEQ ID NO 2): | agcttagtaaagccctcgctag |

A synthetic double-stranded nucleic acid linker comprising the multicloning site and adc transcriptional terminator was used to generate pME101VB01. Two 100 bases oligonucleotides that complement flanked by NcoI or HindIII digested restriction sites were annealed. The 100-base pair product was subcloned into NcoI/HindIII digested plasmid pME101 to generate pME101VB01.

pME101VB01 1, consisting of 100 bases
(SEQ ID NO 3):
catgggctagctacgtattaattaaagatctcctagggagctcaccggtT
AAAAATAAGAGTTACCTTAAATGGTAACTCTTATTTTTTTAggcgcgcca
pME101VB01 2, consisting of 100 bases
(SEQ ID NO 4):
agcttggcgcgccTAAAAAAATAAGAGTTACCATTTAAGGTAACTCTTAT
TTTTAaccggtgagctccctaggagatctttaattaatacgtagctagcc

with:
a region (underlined lower-case letters) corresponding to the multicloning site
a region (upper-case letters) corresponding to the adc transcription terminator (sequence 179847 to 179814) of Clostridium acetobutylicum ATCC 824 pSOL1 (NC_{—}001988).
   Construction of Plasmids for Expression of Different Combinations of Genes of the Biosynthetic Pathway of 1,2-propanediol (pME101VB01-yqhD-mgsA-gldA, pME101VB01-yafB-mgsA-gldA and pME101VB01-yqhE-mgsA-gldA)

The different genes were PCR amplified from genomic DNA of E. coli MG1655 using the oligonucleotides given in Table 1.

TABLE 1
oligonucleotides used for amplification of genes of
1,2-propanediol pathway

| Gene | Names of | | | |
|---|---|---|---|---|
| name | oligos | SEQ ID | Homology with gene | Restriction sites |
| yqhD | yqhDR2 | N^{o} 5 | 3153369-3153400 | BspHI added |
| | yqhDF2 | N^{o} 6 | 3154544-3154475 | BspHI removed |
| | | | | NheI added |
| mgsA | mgsAF | N^{o} 7 | 1026268-1026248 | SnaBI added |
| | mgsAR | N^{o} 8 | 1025780-1025800 | BglII added |
| gldA | gldAF | N^{o} 9 | 4136631-4136612 | AvrII added |
| | gldAR | N^{o} 10 | 4135512-4135530 | SacI added |
| yafB | yafB F2 | N^{o} 11 | 229167-229190 | NcoI added |
| | yafB R | N^{o} 12 | 229970-229950 | NheI added |
| yqhE | yqhE F | N^{o} 13 | 3154641-3154661 | NcoI added |
| | yqhE R | N^{o} 14 | 3155464-3155444 | NheI added |

The PCR amplified fragments were cut with the restriction enzymes mentioned in Table 1 and cloned into the restriction sites of the plasmidpME101VB01. The following plasmids were built: pME101VB01-yqhD-mgsA-gldA, pME101VB01-yafB-mgsA-gldA and pME101VB01-yqhE-mgsA-gldA.

The plasmids were then introduced into the strain E. coli MG1655.

b) Construction of a Modified Strain of E. coliMG1655 Ptrc16-gapA::cm

The replacement of the natural gapA promoter with the synthetic short Ptrc16 promoter (SEQ ID NO 15: gagctgttgacgattaatcatccggctcgaataatgtgtgg) into the strain E. coli MG1655 was made by replacing 225 pb of upstream gapA sequence with FRT-CmR-FRT and an engineered promoter. The technique used was described by Datsenko, K. A. & Wanner, B. L. (2000).

The two oligonucleotides used to replace the natural gapA promoter according to the Protocol 1 are given in Table 2.

Protocol 1: Introduction of a PCR Product for Recombination and Selection of the Recombinants

The oligonucleotides chosen and given in Table 2 for replacement of a gene or an intergenic region were used to amplify either the chloramphenicol resistance cassette from the plasmid pKD3 or the kanamycin resistance cassette from the plasmid pKD4 (Datsenko, K. A. & Wanner, B. L. (2000). The PCR product obtained was then introduced by electroporation into the recipient strain bearing the plasmid pKD46 in which the system Red ( . . . exo) expressed greatly favours homologous recombination. The antibiotic-resistant transformants were then selected and the insertion of the resistance cassette was checked by PCR analysis with the appropriate oligonucleotides given in Table 3.

The resulting strain was named E. coli MG1655 Ptrc16-gapA::cm.

The 3 plasmids were introduced separately into the strain E. coli MG1655 Ptrc16-gapA::cm.

TABLE 2

oligonucleotides used for replacement of a chromosomal region by
recombination with a PCR product in the strain E. coli MG1655

| | | | |
|---|---|---|---|
| Region name | Names of oligos | SEQ ID | Homology with chromosomal region |

| | | | |
|---|---|---|---|
| gapA promoter | Ptrc-gapAF | N^{o}16 | 1860478-1860536 |
| (Ptrc16-gapA) | Ptrc16-gap AR | N^{o}17 | 1860762-1860800 |
| edd and eda genes | DeddF | N^{o}18 | 1932582-1932501 |
| | DedaR | N^{o}19 | 1930144-1930223 |
| gloA gene | GLOAD f | N^{o}20 | 1725861-1725940 |
| | GLOA D R | N^{o}21 | 1726268-1726189 |
| aldA gene | AldA D f | N^{o}22 | 1486256-1486336 |
| | aldAD r | N^{o}23 | 1487695-1487615 |
| aldB gene | AldB D f | N^{o}24 | 3752603-3752682 |
| | aldBD r | N^{o}25 | 3754141-3754062 |
| IdhA gene | DldhAF | N^{o}26 | 1440865-1440786 |
| | DldhAR | N^{o}27 | 1439878-1439958 |
| pflAB gene | DpflB r | N^{o}28 | 952315-952236 |
| | DpflAf | N^{o}29 | 949470-949549 |
| adhE gene | DadhE r | N^{o}30 | 1297344-1297264 |
| | DadhEf | N^{o}31 | 1297694-1297773 |
| ackA-pta genes | DackAF | N^{o}32 | 2411494-2411573 |
| | DptaR | N^{o}33 | 2414906-2414830 |
| poxB gene | DpoxBF | N^{o}34 | 908557-908635 |
| | DpoxBR | N^{o}35 | 910262-910180 |
| pykA gene | DpykAF | N^{o}36 | 1935756-1935836 |
| | DpykAR | N^{o}37 | 1755129-1755051 |
| pykF gene | DpykFF | N^{o}38 | 1753689-1753766 |
| | DpykFR | N^{o}39 | 1755129-1755051 |

TABLE 3

oligonucleotides used for checking the insertion of a
resistance cassette or the loss of a resistance cassette

| | | | |
|---|---|---|---|
| Region name | Names of oligos | SEQ ID | Homology with chromosomal region |

| | | | |
|---|---|---|---|
| gapA promoter | yeaAF | N^{o}40 | 1860259-1860287 |
| (Ptrc16-gapA) | gapAR | N^{o}41 | 1861068-1861040 |
| edd and eda genes | eddF | N^{o}42 | 1932996-1932968 |
| | edaR | N^{o}43 | 1929754-1929777 |
| gloA gene | NemAQd | N^{o}44 | 1725331 to 1725361 |
| | Rnt Cr | N^{o}45 | 1726795 to 1726765 |
| aldA gene | Ydc F C f | N^{o}46 | 1485722 to 1485752 |
| | gapCCr | N^{o}47 | 1488225 to 1488195 |
| aldB gene | aldB C f | N^{o}48 | 3752056 to 3752095 |
| | YiaYCr | N^{o}49 | 3754674 to 3754644 |
| ldhA gene | ldhAF | N^{o}50 | 1439724 to 1439743 |
| | ldhAR | N^{o}51 | 1441029 to 1441007 |
| pflAB gene | pflAB 1 | N^{o}52 | 948462 to 948491 |
| | pflAB 2 | N^{o}53 | 953689 to 983660 |
| adhE | ychGf | N^{o}54 | 1294357 to1294378 |
| | adhECr | N^{o}55 | 1297772 to 1297749 |
| ackA-pta genes | B2295 | N^{o}56 | 2410900 to 2410919 |
| | YfcCR | N^{o}57 | 2415164 to 2415145 |
| poxB gene | poxBF | N^{o}58 | 908475 to 908495 |
| | poxBR | N^{o}59 | 910375 to 910352 |
| pykA gene | pykAF | N^{o}60 | 1935338 to 1935360 |
| | pykAR | N^{o}61 | 1937425 to 1937401 |
| pykF gene | pykFF | N^{o}62 | 1753371 to 1753392 |
| | pykFR | N^{o}63 | 1755518 to 1755495 |

### Example 2

### Construction of Modified Strains of E. coli MG1655 Ptrc16-gapA, Δedd-eda, ΔgloA, ΔpykA, ΔpykF (pME101VB01-yqhD-mgsA-gldA), (pJB137-PgapA-ppsA), E. coli MG1655 Ptrc16-gapA, Δedd-eda, ΔgloA, ΔpykA, ΔpykF (pME101VB01-yafB-mgsA-g/dA), (pJB137-PgapA-ppsA) and E. coli MG1655 Ptrc16-gapA, Δedd-eda, bgloA, ΔpykA, ΔpykF (pME101VB01-yqhE-mgsA-gldA), (pJB137-PgapA-ppsA) able to Produce 1,2-propanediol with High Yield

The genes edd-eda were inactivated in strain E. coli MG1655 by inserting a kanamycin antibiotic resistance cassette and deleting most of the genes concerned using the technique described in Protocol 1 with the oligonucleotides given in Table 2. The strain obtained was named MG1655 Δedd-eda::kin.

This deletion was transferred in strain E. coli MG1655 Ptrc16-gapA::cm according to Protocol 2.

Protocol 2: Transduction with Phage P1 for Deletion of a Gene

The deletion of the chosen gene by replacement of the gene by a resistance cassette (kanamycin or chloramphenicol) in the recipient E. coli strain was performed by the technique of transduction with phage P1. The protocol was in two steps, (i) the preparation of the phage lysate on the strain MG1655 with a single gene deleted and (ii) the transduction of the recipient strain by this phage lysate.

Preparation of the Phage Lysate
Seeding with 100 μl of an overnight culture of the strain MG1655 with a single gene deleted of 10 ml of LB+Cm 30 μg/ml+glucose 0.2%+CaCl₂ 5 mM.
Incubation for 30 min at 37° C. with shaking
Addition of 100 μl of phage lysate P1 prepared on the wild type strain MG1655 (approx. 1×10⁹ phage/ml).
Shaking at 37° C. for 3 hours until all cells were lysed.
Addition of 200 μl of chloroform, and vortexing.
Centrifugation for 10 min at 4500 g to eliminate cell debris.
Transfer of supernatant in a sterile tube and addition of 200 μl of chloroform.
Storage of the lysate at 4° C.

Transduction
Centrifugation for 10 min at 1500 g of 5 ml of an overnight culture of the E. coli recipient strain in LB medium.
Suspension of the cell pellet in 2.5 ml of MgSO₄ 10 mM, CaCl₂ 5 mM.
Control tubes: 100 μl A cells
   100 μl phages P1 of the strain MG1655 with a single gene deletion.
Tube test: 100 μl of cells+100 μl phages P1 of strain MG1655 with a single gene deletion.
Incubation for 30 min at 30° C. without shaking
Addition of 100 μl sodium citrate 1 M in each tube, and vortexing.
Addition of 1 ml of LB.
Incubation for 1 hour at 37° C. with shaking
Plating on dishes LB+Cm 30 μg/ml after centrifugation of tubes for 3 min at 7000 rpm.
Incubation at 37° C. overnight.

The antibiotic-resistant transformants were then selected and the insertion of the deletion was checked by a PCR analysis with the appropriate oligonucleotides.

The resulting strain was named E. coli MG1655 Ptrc16-gapA::cm, Δedd-eda::km.

The antibiotic resistance cassettes were then eliminated according to Protocol 3.

Protocol 3: Elimination of Resistance Cassettes

The chloramphenicol and/or kanamycin resistance cassettes were eliminated according to the following technique. The plasmid pCP20 carrying the FLP recombinase acting at the FRT sites of the chloramphenicol and/or kanamycin resistance cassettes were introduced into the recombinant strains by electroporation. After serial culture at 42° C., the loss of the antibiotics resistance cassettes was checked by PCR analysis with the oligonucleotides given in Table 3.

The strain MG1655 ΔgloA::cm was built according to Protocol 1 with the oligonucleotides given in Table 2 and this deletion was transferred in the strain previously built according to Protocol 2. The resulting strain was named E. coli MG1655 Ptrc16-gapA, Δedd-eda,ΔgloA::cm.

The gene pykA was inactivated into the previous strain by inserting a kanamycin antibiotic resistance cassette according to Protocol 1 with the oligonucleotides given in Table 2. The resulting strain was named E. coli MG1655 Ptrc16-gapA, Δedd-eda,ΔgloA::cm, ΔpykA::km.

The antibiotic resistance cassettes were then eliminated according to Protocol 3.

The gene pykF was inactivated by inserting a chloramphenicol antibiotic resistance cassette according to Protocol 1 with the oligonucleotides given in Table 2. The resulting strain was named E. coli MG1655 Ptrc16-gapA, Δedd-eda,ΔgloA, ΔpykA, ΔpykF::cm.

The antibiotic resistance cassette was then eliminated according to Protocol 3.

At each step, the presence of all the deletions previously built was checked using the oligonucleotides given in Table 3.

To increase the production of phosphoenolpyruvate the ppsA gene was expressed from the plasmid pJB 137 using the gapA promoter. For the construction of plasmid pJB137-PgapA-ppsA, the gene ppsA was PCR amplified from genomic DNA of E. coli MG1655 using the following oligonucleotides:

1. gapA-ppsAF, consisting of 65 bases

(SEQ ID NO 64)

ccttttattcactaacaaatagctggtggaatatATGTCCAACAATGGCT
CGTCACCGCTGGTGC

with:
a region (upper-case letters) homologous to the sequence (1785106-1785136) of the gene ppsA (1785136 to 1782758), a reference sequence on the website http://genolist.pasteurfr/Colibri/), and
a region (lower letters) homologous to the gapA promoter (1860794-1860761).

2. ppsAR, consisting of 43 bases

(SEQ ID NO 65)

aatcgcaagcttGAATCCGGTTATTTCTTCAGTTCAGCCAGGC

with:
a region (upper letters) homologous to the sequence (1782758-1782780) the region of the gene ppsA (1785136 to 1782758)
a restriction site HindIII (underlined letters)

At the same time the gapA promoter region of the E. coli gene gapA was amplified using the following oligonucleotides:

1. gapA-ppsAR, consisting of 65 bases

(SEQ ID NO 66)

GCACCAGCGGTGACGAGCCATTGTTGGACATatattccaccagctatttg
ttagtgaataaaagg

with:
a region (upper-case letters) homologous to the sequence (1785106-1785136) of the gene ppsA (1785136 to 1782758), and
a region (lower letters) homologous to the gapA promoter (1860794-1860761).

| | |
|---|---|
| 2. gapAF, consisting of 33 bases | |
| ACGTCCCGGGcaagcccaaaggaagagtgaggc | (SEQ ID NO 67) |

with:
a region (lower letters) homologous to the gapA promoter (1860639-1860661).
a restriction site SmaI (underlined letters)

Both fragments were subsequently fused using the oligonucleotides ppsAR and gapAF (Horton et al. 1989 Gene 77:61-68). The PCR amplified fragment were cut with the restriction enzymes HindIII and SmaI and cloned into the HindIII SmaI sites of the vector pJ/3137 (EMBL Accession number: U75326) giving vector pJB137-PgapA-ppsA.

The different pME101VB01 plasmids and pJB137-PgapA-ppsA were introduced into the strain E. coli MG1655 Ptrc16-gapA, Δedd-eda,ΔgloA, ΔpykA, ΔpykF. The strains obtained were named respectively E. coli MG1655 Ptrc16-gapA, Δedd-eda,ΔgloA, ΔpykA, ΔpykF, pME101VB01-yqhD-mgsA-gldA, pJB137-PgapA-ppsA (strain 1), E. coli MG1655 Ptrc16-gapA, Δedd-eda,ΔgloA, ΔpykA, ΔpykF, pME101VB01-yafB-mgsA-gldA, pJB137-PgapA-ppsA (strain 2) and E. coli MG1655 Ptrc16-gapA, Δedd-eda, ΔgloA, ΔpykA, ΔpykF, pME101VB01-yqhE-mgsA-gldA, pJB137-PgapA-ppsA (strain 3).

### Example 3

### Construction of a Modified Strains of E. coli MG1655 Ptrc16-gapA, Δedd-eda, ΔgloA, ΔaldA, ΔaldB, ΔldhA, ΔpflAB, ΔadhE, ΔackA-pta, ΔpoxB, ΔpykA, ΔpykF (pME101VB01-yqhD-mgsA-g/dA), (pJB137-PgapA-ppsA), E. coli MG1655 Ptrc16-gapA, Δedd-eda, ΔgloA, ΔaldA, ΔaldB, ΔldhA, ΔpflAB, ΔadhE, ΔackA-pta, ΔpoxB, ΔpykA, ΔpykF (pME101VB01-yafB-mgsA-g/dA), (pJB137-PgapA-ppsA) and E. coli MG1655 Ptrc16-gapA, Δedd-eda, ΔgloA, ΔaldA, ΔaldB, ΔldhA, ΔpflAB, ΔadhE, ΔackA-pta, ΔpoxB, ΔpykA, ΔpykF (pME101VB01-yqhE-mgsA-g/dA), (pJB137-PgapA-ppsA) able to produce 1,2-propanediol with a Yield Higher than 1 Mole/Mole Glucose

The strains MG1655 ΔaldA::km, MG1655 ΔaldB::cm, MG1655 ΔpflAB::km MG1655 ΔadhE::cm, MG1655 ΔackA-pta::cm are built according to Protocol 1 with the oligonucleotides given in Table 2 and these deletions are transferred in the strain previously built according to Protocol 2. When necessary, the antibiotic resistance cassettes are eliminated according to Protocol 3.

The gene ldhA and the gene poxB are inactivated in the strain previously built by inserting a chloramphenicol antibiotic resistance cassette according to Protocol 1 with the oligonucleotides given in Table 2. When necessary, the antibiotic resistance cassettes are eliminated according to Protocol 3.

At each step, the presence of all the deletions previously built is checked using the oligonucleotides given in Table 3.

The resulting strain is named E. coli MG1655 Ptrc16-gapA, Δedd-eda,ΔgloA, ΔaldA, ΔaldB, ΔldhA, ΔpflAB, ΔadhE, ΔackA-pta, ΔpoxB, ΔpykA, ΔpykF.

The differents pME101VB01 plasmids and pJB137-PgapA-ppsA are introduced into the strain E. coli MG1655 Ptrc16-gapA, Δedd-eda,ΔgloA, ΔaldA,ΔaldB, ΔldhA, ΔpflAB, ΔadhE, ΔackA-pta, ΔpoxB, ΔpykA, ΔpykF. The strains obtained are named respectively E. coli MG1655 Ptrc16-gapA, Δedd-eda,ΔgloA, ΔaldA,ΔaldB, ΔldhA, ΔpflAB, ΔadhE, ΔackA-pta, ΔpoxB, ΔpykA, ΔpykF, pME101VB01-yqhD-mgsA-gldA, pJB137-PgapA-ppsA, E. coli MG1655 Ptrc16-gapA, Δedd-eda,ΔgloA, ΔaldA,ΔaldB, ΔldhA, ΔpflAB, ΔadhE, ΔackA-pta, ΔpoxB, ΔpykA, ΔpykF, pME101VB01-yafB-mgsA-gldA, pJB137-PgapA-ppsA and E. coli MG1655 Ptrc16-gapA, Δedd-eda,ΔgloA, ΔaldA,ΔaldB, ΔldhA, ΔpflAB, ΔadhE, ΔackA-pta, ΔpoxB, ΔpykA, ΔpykF, pME101VB01-yqhE-mgsA-gldA, pJB137-PgapA-ppsA.

### Example 4

### Comparison of the Different Strains for 1,2-propanediol production Under Aerobic Conditions

The strains obtained as described in example 2 (strains 1, 2 and 3) and the control strains (control 1: MG1655 pME101VB01-yqhD-mgsA-gldA, control 2: MG1655 pME101VB01-yafB-mgsA-gldA, control 3: MG1655 pME101VB01-yqhE-mgsA-gldA and control 4: MG1655 Ptrc16-gapA, Δedd-eda,ΔgloA, ΔpykA, ΔpykF) were cultivated in an Erlenmeyer flask assay under aerobic conditions in minimal medium with glucose as carbon source. The culture was carried out at 34° C. or 37° C. and the pH was maintained by buffering the culture medium with MOPS. At the end of the culture, 1,2-propanediol, acetol and residual glucose in the fermentation broth were analysed by HPLC and the yields of 1,2-propanediol over glucose and 1,2-propanediol+acetol over glucose were calculated. The best strain is then selected for a fermenter fed-batch culture.

| | 1,2- | | 1,2- | 1,2- |
|---|---|---|---|---|
| | propanediol | | propanediol | propanediol + |
| | titer | Acetol | yield | acetol yield (g/g |
| Strain | (g/l) | titer (g/l) | (g/g glucose) | glucose) |
| Control 1 | 0.02 | 0 | 0.004 | 0.004 |
| Control 2 | 0 | 0 | 0 | 0 |
| Control 3 | 0.01 | 0 | 0.002 | 0.002 |
| Control 4 | 0.05 | 0.34 | 0 | 0.04 |
| Strain 1 | 2.25 | 1.40 | 0.14 | 0.23 |
| Strain 2 | 1.64 | 1.31 | 0.10 | 0.18 |
| Strain 3 | 0.77 | 0.47 | 0.06 | 0.10 |

### Example 5

### Production of 1,2-propanediol in Fed-Batch Culture with the Best Strain

The best strain selected in the previous experiment is cultivated in a 21 fermenter using a fed-batch protocol.

The temperature of the culture is maintained constant at 37° C. and the pH is permanently adjusted to values between 6.5 and 8 using an NH₄OH solution. The agitation rate is maintained between 200 and 300 rpm during the batch phase and is increased to up to 1000 rpm at the end of the fed-batch phase. The concentration of dissolved oxygen is maintained at values between 30 and 40% saturation by using a gas controller. When the optical density reaches a value between three and five, the fed-batch is started with an initial flow rate between 0.3 and 0.5 ml/h and a progressive increase up to flow rate values between 2.5 and 3.5 ml/h. At this point the flow rate is maintained constant for 24 to 48 hours. The medium of the fed is based on minimal media containing glucose at concentrations between 300 and 500 g/l.

1. Microorganism useful for the production of 1,2-propanediol from a carbon source, wherein said microorganism is characterized by:
an improved activity of the biosynthesis pathway from dihydroxyacetone phosphate to 1,2-propanediol, and
an attenuated activity of the glyceraldehyde 3-phosphate dehydrogenase.

2. The microorganism according to claim 1 wherein it is genetically modified to increase the activity of at least one enzyme involved in the biosynthesis pathway from dihydroxyacetone phosphate to 1,2-propanediol.

3. The microorganism according to claim 2 wherein the increase of the activity of at least one enzyme is obtained by increasing the expression of the gene coding for said enzyme.

4. The microorganism according to claim 3 wherein the expression of at least one gene selected among the group consisting of: mgsA, yafB, yeaE, yghZ, yqhE, yqhD, ydhF, ycdW, yajO, ydjG, ydbC, tas, gldA and fucO is increased.

5. The microorganism according to claim 4 wherein the expression of three genes mgsA, yqhD and gldA is increased.

6. The microorganism according to anyone of claims 1 to 5 wherein the activity of at least one enzyme involved in the Entner-Doudoroff pathway is attenuated.

7. The microorganism according to claim 6 wherein the expression of at least one of the following genes is attenuated: edd, eda.

8. The microorganism according to anyone of claims 1 to 7 wherein the activity of at least one enzyme involved in the conversion of methylglyoxal into lactate is attenuated.

9. The microorganism according to claim 8 wherein the expression of at least one of the following genes is attenuated: gloA, aldA, aldB.

10. The microorganism according to claims 1 to 9 wherein the activity of at least one enzyme involved in the synthesis of lactate, formate or ethanol is attenuated.

11. The microorganism according to claim 10 wherein the expression at least one of the following genes is attenuated: ldhA, pflA, pflB, adhE.

12. The microorganism according to anyone of claims 1 to 11 wherein the activity of at least one enzyme involved in the synthesis of acetate is attenuated.

13. The microorganism according to claim 12 wherein the expression of at least one of the following gene is attenuated: ackA, pta, poxB.

14. The microorganism according to claims 1 to 13 wherein the efficiency of the sugar import is increased.

15. The microorganism according to claim 14 wherein a sugar import system independent of phosphoenolpyruvate is used.

16. The microorganism according to claim 15 wherein the expression of at least one gene selected among galP and glk is increased.

17. The microorganism according to claim 14 wherein the efficiency of the sugar-phosphotransferase system is improved by increasing the availability of the metabolite ‘phosphoenolpyruvate’.

18. The microrganism according to claim 17 wherein the activity of at least one enzyme pyruvate kinase is attenuated.

19. The microorganism according to claim 18 wherein the expression of at least one gene selected among pykA and pykF is attenuated.

20. The microrganism according to anyone of claims 17 to 19 wherein the phosphoenolpyruvate synthase activity is increased.

21. The microorganism according to claim 20 wherein the expression of the ppsA gene is increased.

22. The microorganism according to anyone of claims 1 to 21 wherein the enzyme that favours the metabolism of pyruvate into acetyl-CoA has lower sensitivity to the inhibition by NADH than the unmodified enzyme.

23. The microorganism according to claim 22 wherein the gene lpd has a point mutation leading to the replacement of alanine 55 with valine.

24. The microorganism according to anyone of claims 1 to 23 wherein the expression of at least one gene selected among arcA and ndh is attenuated.

25. A microorganism according to anyone of claims 1 to 24 wherein the microorganism is selected from the group consisting of bacteria, yeasts and fungi.

26. The microorganism according to claim 25 wherein the microorganism is selected from the group consisting of Enterobacteriaceae, Bacillaceae, Clostridiaceae, Streptomycetaceae and Corynebacteriaceae.

27. The microorganism according to claim 26 wherein the microorganism is either Escherichia coli or Clostridium acetobutylicum.

28. A method for preparing 1,2-propanediol wherein a microorganism according to anyone of claims 1 to 27 is grown in an appropriate growth medium containing a carbon source, and the produced 1,2-propanediol is recovered.

29. The method according to claim 28 wherein the microorganism is Escherichia coli and the carbon source is a simple carbon source.

30. The method according to claim 28 wherein the microorganism is Clostridium acetobutylicum and the carbon source is a complex carbon source.

31. The method according to anyone of claims 28 to 30, wherein the recovered 1,2-propanediol is furthermore purified.


SEQUENCE LISTING (67 sequences)

SEQ ID NO: 1 - DNA, 24 bp, Artificial
  PCR primer
ccgacagtaagacgggtaagcctg

SEQ ID NO: 2 - DNA, 22 bp, Artificial
  PCR primer
agcttagtaaagccctcgctag

SEQ ID NO: 3 - DNA, 100 bp, Artificial
  PCR primer
catgggctagctacgtattaattaaagatctcctagggagctcaccggttaaaaataagagttaccttaaatggtaactcttatttttttaggcgcgcca

SEQ ID NO: 4 - DNA, 100 bp, Artificial
  PCR primer
agcttggcgcgcctaaaaaaataagagttaccatttaaggtaactcttatttttaaccggtgagctccctaggagatctttaattaatacgtagctagcc

SEQ ID NO: 5 - DNA, 43 bp, Artificial
  PCR primer
cgatgcacgtcatgaacaactttaatctgcacaccccaacccg

SEQ ID NO: 6 - DNA, 79 bp, Artificial
  PCR primer
ctagctagcggcgtaaaaagcttagcgggcggcttcgtatatacggcggctgacatccaacgtaatgtcgtgattttcg

SEQ ID NO: 7 - DNA, 29 bp, Artificial
  PCR primer
cgtacgtactgtaggaaagttaactacgg

SEQ ID NO: 8 - DNA, 29 bp, Artificial
  PCR primer
gaagatctttacttcagacggtccgcgag

SEQ ID NO: 9 - DNA, 28 bp, Artificial
  PCR primer
gacctaggctctaaaggagcaattatgg

SEQ ID NO: 10 - DNA, 26 bp, Artificial
  PCR primer
cgagctcttattcccactcttgcagg

SEQ ID NO: 11 - DNA, 30 bp, Artificial
  PCR primer
catgccatggctatccctgcatttggttta

SEQ ID NO: 12 - DNA, 30 bp, Artificial
  PCR primer
ctagctagcttaatcccattcaggagccag

SEQ ID NO: 13 - DNA, 31 bp, Artificial
  PCR primer
catgccatggctaatccaaccgttattaagc

SEQ ID NO: 14 - DNA, 30 bp, Artificial
  PCR primer
ctagctagcttagccgccgaactggtcagg

SEQ ID NO: 15 - DNA, 41 bp, Artificial
  Synthetic promoter
gagctgttgacgattaatcatccggctcgaataatgtgtgg

SEQ ID NO: 16 - DNA, 100 bp, Artificial
  PCR primer
agtcatatattccaccagctatttgttagtgaataaaagccacacattattcgagccggatgattaatagtcaacagctctgtaggctggagctgcttcg

SEQ ID NO: 17 - DNA, 79 bp, Artificial
  PCR primer
gctcacattacgtgactgattctaacaaaacattaacaccaactggcaaaattttgtcccatatgaatatcctccttag

SEQ ID NO: 18 - DNA, 102 bp, Artificial
  PCR primer
cgcgcgagactcgctctgcttatctcgcccggatagaacaagcgaaaacttcgaccgttcatcgttcgcagttggcatgcggtgtaggctggagctgcttcg

SEQ ID NO: 19 - DNA, 100 bp, Artificial
  PCR primer
gcttagcgccttctacagcttcacgcgccagcttagtaatgcggtcgtaatcgcccgcttccagcgcatctgccggaacccatatgaatatcctccttag

SEQ ID NO: 20 - DNA, 101 bp, Artificial
  PCR primer
atgcgtcttcttcataccatgctgcgcgttggcgatttgcaacgctccatcgatttttataccaaagtgctgggcatgaagtgtaggctggagctgcttcg

SEQ ID NO: 21 - DNA, 100 bp, Artificial
  PCR primer
ttagttgcccagaccgcgaccggcgtctttctcttcgattaactcaattttgtaaccgtccggatcttccacaaacgcgacatatgaatatcctccttag

SEQ ID NO: 22 - DNA, 102 bp, Artificial
  PCR primer
atgtcagtacccgttcaacatcctatgtatatcgatggacagtttgttacctggcgtggagacgcatggattgatgtggtagtgtaggctggagctgcttcg

SEQ ID NO: 23 - DNA, 101 bp, Artificial
  PCR primer
ttaagactgtaaataaaccacctgggtctgcagatattcatgcaagccatgtttaccatctgcgccgccaataccggatttcatatgaatatcctccttag

SEQ ID NO: 24 - DNA, 101 bp, Artificial
  PCR primer
tcagaacagccccaacggtttatccgagtagctcaccagcaggcacttggtttgctggtaatgctccagcatcatcttgtgtgtaggctggagctgcttcg

SEQ ID NO: 25 - DNA, 100 bp, Artificial
  PCR primer
atgaccaataatcccccttcagcacagattaagcccggcgagtatggtttccccctcaagttaaaagcccgctatgacaacatatgaatatcctccttag

SEQ ID NO: 26 - DNA, 100 bp, Artificial
  PCR primer
gaaactcgccgtttatagcacaaaacagtacgacaagaagtacctgcaacaggtgaacgagtcctttggctttgagctggtgtaggctggagctgcttcg

SEQ ID NO: 27 - DNA, 101 bp, Artificial
  PCR primer
ttaaaccagttcgttcgggcaggtttcgcctttttccagattgcttaagttttgcagcgtagtctgagaaatactggtcagcatatgaatatcctccttag

SEQ ID NO: 28 - DNA, 100 bp, Artificial
  PCR primer
ccggacatcctgcgttgccgtaaatctggtgttctgaccggtctgccagatgcatatggccgtggccgtatcatcggtgacatatgaatatcctccttag

SEQ ID NO: 29 - DNA, 100 bp, Artificial
  PCR primer
gatgcactataagatgtgttaaaaacgctgtagcagaatgaagcgcggaataaaaaagcggcaactcaataaagttgccgtgtaggctggagctgcttcg

SEQ ID NO: 30 - DNA, 101 bp, Artificial
  PCR primer
atggctgttactaatgtcgctgaacttaacgcactcgtagagcgtgtaaaaaaagcccagcgtgaatatgccagtttcactcatatgaatatcctccttag

SEQ ID NO: 31 - DNA, 100 bp, Artificial
  PCR primer
caataacgaatgatagcaattttaagtagttaggaggtgaaaaatgctgtcaaaaggcgtattgtcagcgcgtcttttcatgtaggctggagctgcttcg

SEQ ID NO: 32 - DNA, 100 bp, Artificial
  PCR primer
cgagtaagttagtactggttctgaactgcggtagttcttcactgaaatttgccatcatcgatgcagtaaatggtgaagagtgtaggctggagctgcttcg

SEQ ID NO: 33 - DNA, 97 bp, Artificial
  PCR primer
gctgctgtgcagactgaatcgcagtcagcgcgatggtgtagacgatatcgtcaaccagtgcgccacgggacaggtcgcatatgaatatcctccttag

SEQ ID NO: 34 - DNA, 99 bp, Artificial
  PCR primer
ccttagccagtttgttttcgccagttcgatcacttcatcaccgcgtccgctgatgattgcgcgcagcatatacaggctgcatatgaatatcctccttag

SEQ ID NO: 35 - DNA, 102 bp, Artificial
  PCR primer
cggttgcagcttatatcgccaaaacactcgaatcggcaggggtgaaacgcatctggggagtcacaggcgactctctgaacggtgtaggctggagctgcttcg

SEQ ID NO: 36 - DNA, 101 bp, Artificial
  PCR primer
cgcggcgggtgccaacgttgtacgtatgaacttttctcacggctcgcctgaagatcacaaaatgcgcgcggataaagttcgtgtaggctggagctgcttcg

SEQ ID NO: 37 - DNA, 101 bp, Artificial
  PCR primer
cgccgcatccggcaacgtacttactctaccgttaaaatacgcgtggtattagtagaacccacggtactcatcacgtcgccccatatgaatatcctccttag

SEQ ID NO: 38 - DNA, 98 bp, Artificial
  PCR primer
cccatccttctcaacttaaagactaagactgtcatgaaaaagaccaaaattgtttgcaccatcggaccgaaaaccgaatgtaggctggagctgcttcg

SEQ ID NO: 39 - DNA, 99 bp, Artificial
  PCR primer
ggacgtgaacagatgcggtgttagtagtgccgctcggtaccagtgcaccagaaaccataactacaacgtcacctttgtgcatatgaatatcctccttag

SEQ ID NO: 40 - DNA, 29 bp, Artificial
  PCR primer
gccacagccggaatcatacttggtttggg

SEQ ID NO: 41 - DNA, 29 bp, Artificial
  PCR primer
cgtcaacaccaacttcgtcccatttcagg

SEQ ID NO: 42 - DNA, 29 bp, Artificial
  PCR primer
gggtagactccattactgaggcgtgggcg

SEQ ID NO: 43 - DNA, 24 bp, Artificial
  PCR primer
ccacatgataccgggatggtgacg

SEQ ID NO: 44 - DNA, 31 bp, Artificial
  PCR primer
gaagtggtcgatgccgggattgaagaatggg

SEQ ID NO: 45 - DNA, 31 bp, Artificial
  PCR primer
gggttacgtttcagtgaggcgcgttctgcgg

SEQ ID NO: 46 - DNA, 31 bp, Artificial
  PCR primer
tgcagcggcgcacgatggcgacgttccgccg

SEQ ID NO: 47 - DNA, 31 bp, Artificial
  PCR primer
cacgatgacgaccattcatgcctatactggc

SEQ ID NO: 48 - DNA, 40 bp, Artificial
  PCR primer
catatttccctcaaagaatataaaaaagaacaattaacgc

SEQ ID NO: 49 - DNA, 31 bp, Artificial
  PCR primer
tatgttcatgcgatggcgcaccagctgggcg

SEQ ID NO: 50 - DNA, 20 bp, Artificial
  PCR primer
gccatcagcaggcttagcgc

SEQ ID NO: 51 - DNA, 23 bp, Artificial
  PCR primer
gggtattgtggcatgtttaaccg

SEQ ID NO: 52 - DNA, 30 bp, Artificial
  PCR primer
agacattaaaaatatacgtgcagctacccg

SEQ ID NO: 53 - DNA, 30 bp, Artificial
  PCR primer
gtgaaagctgacaacccttttgatctttta

SEQ ID NO: 54 - DNA, 22 bp, Artificial
  PCR primer
ggctcattgcaccaccatccag

SEQ ID NO: 55 - DNA, 24 bp, Artificial
  PCR primer
gaaaagacgcgctgacaatacgcc

SEQ ID NO: 56 - DNA, 20 bp, Artificial
  PCR primer
gcatgggtaaacttaaggcg

SEQ ID NO: 57 - DNA, 20 bp, Artificial
  PCR primer
taatcaccaacgtatcgggc

SEQ ID NO: 58 - DNA, 21 bp, Artificial
  PCR primer
cgcggcttggtcgggtaacgg

SEQ ID NO: 59 - DNA, 24 bp, Artificial
  PCR primer
tcgggctatttaaccgttagtgcc

SEQ ID NO: 60 - DNA, 23 bp, Artificial
  PCR primer
ggcaattaccctcgacgtaccgg

SEQ ID NO: 61 - DNA, 25 bp, Artificial
  PCR primer
ccgatggatgatctgttagaggcgg

SEQ ID NO: 62 - DNA, 22 bp, Artificial
  PCR primer
gcgtaaccttttccctggaacg

SEQ ID NO: 63 - DNA, 24 bp, Artificial
  PCR primer
gcgttgctggagcaacctgccagc

SEQ ID NO: 64 - DNA, 65 bp, Artificial
  PCR primer
ccttttattcactaacaaatagctggtggaatatatgtccaacaatggctcgtcaccgctggtgc

SEQ ID NO: 65 - DNA, 43 bp, Artificial
  PCR primer
aatcgcaagcttgaatccggttatttcttcagttcagccaggc

SEQ ID NO: 66 - DNA, 65 bp, Artificial
  PCR primer
gcaccagcggtgacgagccattgttggacatatattccaccagctatttgttagtgaataaaagg

SEQ ID NO: 67 - DNA, 33 bp, Artificial
  PCR primer
acgtcccgggcaagcccaaaggaagagtgaggc